(19) United States
(12) Patent Application Publication
(10) Pub. No.: US 2004/0180890 A1
(43) Pub. Date: Sep. 16, 2004

(54) Novel piperidinecarboxamide derivatives, method for preparing same and pharmaceutical compositions containing same

(76) Inventors: Xavier Emonds-Alt, Combaillaux (FR); Vincenzo Proietto, Saint Georges D'Orques (FR)

Correspondence Address: Sanofi Synthelabo Inc, 9 Great Valley Parkway, PO Box 3026, Malvern, PA, 19355, US

(21) Appl. No.: 10/477,682

(22) PCT Filed: May 17, 2002
(86) PCT No.: PCT/FR02/01663

(30) Foreign Application Priority Data
May 21, 2001 (FR) 0106691

(51) Int. Cl. A61K 31/5377; C07D 413/14

(52) U.S. Cl. 514/235.2; 544/130

(57) ABSTRACT

The invention relates to compounds of formula:

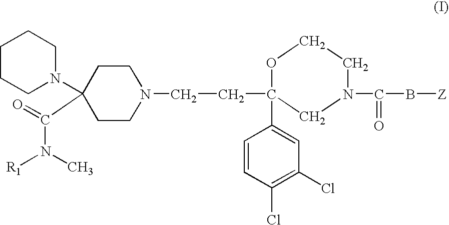

as well as their salts with inorganic or organic acids, their solvates and/or their hydrates, which exhibit a very high affinity both for the human NK₂ receptors for neurokinin A and for the human NK₃ receptors for neurokinin B and are antagonists of the said receptors.

The invention also relates to their method of preparation, the pharmaceutical compositions containing them and their use for the preparation of medicaments.

The subject of the present invention is novel piperidinecarboxamide derivatives, a method for their preparation and pharmaceutical compositions containing them as active ingredient.

More particularly, the present invention relates to novel piperidinecarboxamide derivatives for therapeutic use, in pathological phenomena which involve the tachykinin system such as, for example, without limitation: pain (L. Urban et al., TINS, 1994, 17, 432-438; L. Seguin et al., Pain, 1995, 61, 325-343; S. H. Buck, 1994, The Tachykinin Receptors, Humana Press, Totowa, N.J.), allergy and inflammation (S. H. Buck, 1994, The Tachykinin Receptors, Humana Press, Totowa, N.J.), gastrointestinal disorders (P. Holzer and U. Holzer-Petsche, Pharmacol. Ther., 1997, 73, 173-217 and 219-263), respiratory disorders (J. Mizrahi et al., Pharmacology, 1982, 25, 39-50; C. Advenier et al., Eur. Respir. J., 1997, 10, 1892-1906; C. Advenier and X. Emonds-Alt, Pulmonary Pharmacol., 1996, 9, 329-333), urinary disorders (S. H. Buck, 1994, The Tachykinin Receptors, Humana Press, Totowa, N.J.; C. A. Maggi, Progress in Neurobiology, 1995, 45, 1-98), neurological disorders, neuropsychiatric disorders (C. A. Maggi et al., J. Autonomic Pharmacol., 1993, 13, 23-93; M. Otsuka and K. Yoshioka, Physiol. Rev. 1993, 73, 229-308).

In recent years, numerous research studies have been carried out on tachykinins and their receptors. Tachykinins are distributed both in the central nervous system and in the peripheral nervous system. The tachykinin receptors have been recognized and are classified into three types: NK₁, NK₂, NK₃. Substance P (SP) is the endogenous ligand for the NK₁ receptors, neurokinin A (NKA) that for the NK₂ receptors and neurokinin B (NKB) that for the NK₃ receptors.

The NK₁, NK₂ and NK₃ receptors have been demonstrated in various species.

A review by C. A. Maggi et al. (J. Autonomic Pharmacol., 1993, 13, 23-93) and a review by D. Regoli et al. (Pharmacol. Rev., 1994, 46, 551-599) sum up the tachykinin receptors and their antagonists and disclose the pharmacological studies and the applications in human therapy.

Numerous patents or patent applications describe compounds which are active on the tachykinin receptors. Thus, international application WO 96/23787 relates to the compounds of formula:

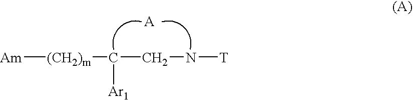

in which:

A may represent the bivalent radical —O—CH₂—CH₂—;

Am, m, Ar₁ and T have different values.

In particular, 1-[2-[4-benzoyl-2-(3,4-dichlorophenyl)morpholin-2-yl]ethyl]-4-(piperidin-1-yl)piperidine-4-carboxamide (compound α) is described in Example 65 of WO 96/23787.

This compound has a high affinity for the human NK₂ receptors but a lower affinity for the human NK₃ receptors.

Patent application EP-A-0 776 893 relates to the compounds of formula:

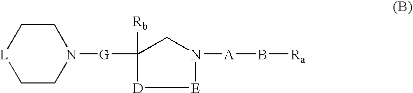

in which in particular:

D-E may represent a bivalent radical —O—CH₂—CH₂—;

L, G, E, A, B, Rₐ and R_{b} have different values.

Patent WO 00/34274 relates to cyclohexylpiperidine derivatives which are antagonists both of the NK₁ receptors for substance P and of the NK₂ receptors for neurokinin A.

Novel compounds have now been found which have a very high affinity both for the human NK₂ receptors for neurokinin A and for the human NK₃ receptors for neurokinin B and which are antagonists of the said receptors.

Furthermore, the compounds according to the present invention have good bioavailability when they are administered by the oral route.

These compounds may be used for the preparation of medicaments useful in the treatment of any pathology where either neurokinin A and/or NK₂ receptors, or neurokinin B and/or NK₃ receptors, or both neurokinin A and neurokinin B and/or NK₂ and NK₃ receptors are involved, in particular in the treatment of pathologies of the respiratory, gastrointestinal, urinary, immune, cardiovascular and central nervous systems as well as in the treatment of pain, migraine, inflammation, nausea and vomiting, and skin diseases.

Thus, according to one of its aspects, the subject of the present invention is compounds of formula:

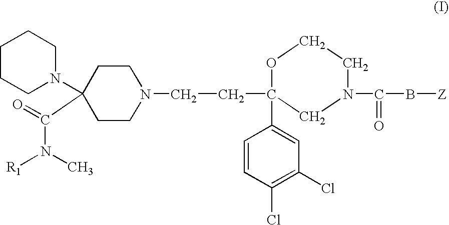

in which:

R₁ represents a hydrogen atom or a methyl radical;

B represents a direct bond or a —CH₂— group;

Z represents a phenyl, a 2,3-dichlorophenyl or a 2,6-dichlorophenyl;

as well as their salts with inorganic or organic acids, their solvates and/or their hydrates.

The compounds of formula (I) according to the invention comprise both the optically pure isomers and mixtures thereof in any proportions.

It is thus possible to form salts of the compounds of formula (I). These salts comprise both those with inorganic or organic acids which allow appropriate separation or crystallization of the compounds of formula (I), such as picric acid or oxalic acid or an optically active acid, for example, a mandelic or camphorsulphonic acid, and those which form pharmaceutically acceptable salts, such as the hydrochloride, hydrobromide, sulphate, hydrogen sulphate, dihydrogen phosphate, methanesulphonate, methyl sulphate, oxalate, maleate, fumarate, succinate, naphthalene-2-sulphonate, gluconate, citrate, isethionate, benzenesulphonate, para-toluenesulphonate, acetate.

The expression halogen atom is understood to mean a chlorine, bromine, fluorine or iodine atom.

According to the present invention, the compounds of formula (I) in the form of optically pure isomers are preferred.

The following compounds:

N,N-dimethyl-1-[2-[4-benzoyl-2-(3,4-dichlorophenyl)-morpholin-2-yl]ethyl]-4-(piperidin-1-yl)piperidine-4-carboxamide, dextrorotatory isomer;

N-methyl-1-[2-[4-benzoyl-2-(3,4-dichlorophenyl)-morpholin-2-yl]ethyl]-4-(piperidin-1-yl)piperidine-4-carboxamide, dextrorotatory isomer;

N,N-dimethyl-1-[2-[4-(2,3-dichlorobenzoyl)-2-(3,4-dichlorophenyl)morpholin-2-yl]ethyl]-4-(piperidin-1-yl)piperidine-4-carboxamide, laevorotatory isomer;

N,N-dimethyl-1-[2-[4-(2,6-dichlorophenyl)acetyl]-2-(3,4-dichlorophenyl)morpholin-2-yl]ethyl]-4-(piperidin-1-yl)piperidine-4-carboxamide, dextrorotatory isomer;

N,N-dimethyl-1-[2-[4-[2-(2,3-dichlorophenyl)acetyl]-2-(3,4-dichlorophenyl)morpholin-2-yl]ethyl]-4-(piperidin-1-yl)piperidine-4-carboxamide, dextrorotatory isomer;

N-methyl-1-[2-[4-[2-(2,3-dichlorophenyl)acetyl]-2-(3,4-dichlorophenyl)morpholin-2-yl]ethyl]-4-(piperidin-1-yl)piperidine-4-carboxamide, dextrorotatory isomer;

as well as their salts with inorganic or organic acids, their solvates and/or their hydrates, are preferred.

The following compound:

N,N-dimethyl-1-[2-[4-benzoyl-2-(3,4-dichlorophenyl)-morpholin-2-yl]ethyl]-4-(piperidin-1-yl)piperidine-4-carboxamide, dextrorotatory isomer;

as well as its salts with inorganic or organic acids, its solvates and/or its hydrates is particularly preferred.

According to another of its aspects, the present invention relates to a method for preparing the compounds of formula (I), their salts, their solvates and/or their hydrates, characterized in that:

a compound of formula:

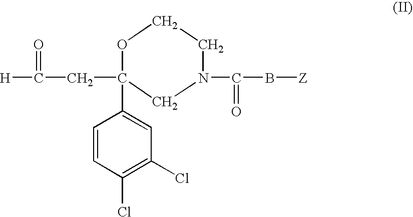

in which B and Z are as defined for a compound of formula (I), is reacted with a compound of formula:

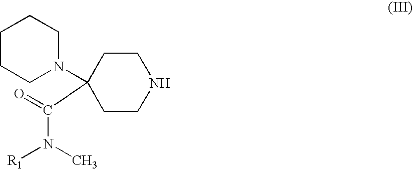

in which R₁ is as defined for a compound of formula (I), in the presence of an acid, in a solvent, and then the intermediate iminium salt formed is reduced by means of a reducing agent.

Optionally, the compound of formula (I) is converted to one of its salts with inorganic or organic acids.

The reaction is carried out in the presence of an acid such as acetic acid, in a solvent such as methanol or dichloromethane, at a temperature between room temperature and the reflux temperature of the solvent, and forms in situ an intermediate imine which is chemically reduced using, for example, sodium cyanoborohydride or sodium triacetoxyborohydride or catalytically using hydrogen and a catalyst such as palladium on carbon or Raney® nickel.

According to a variant of the method:

a compound of formula:

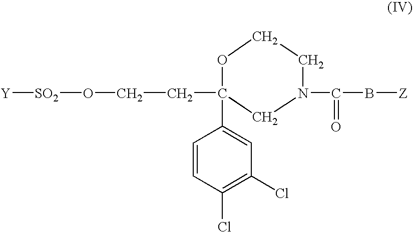

in which B and Z are as defined for a compound of formula (I) and Y represents a methyl, phenyl, tolyl or trifluoromethyl group, is reacted with a compound of formula:

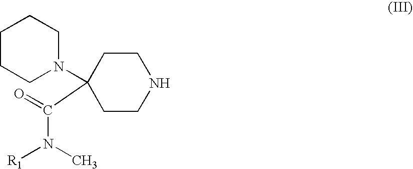

in which R₁ is as defined for a compound of formula (I).

Optionally, the compound of formula (I) is converted to one of its salts with inorganic or organic acids.

The reaction is carried out in an inert solvent such as N,N-dimethylformamide, acetonitrile, methylene chloride, toluene or isopropanol and in the presence or the absence of a base. When a base is used, it is chosen from organic bases such as triethylamine, N,N-diisopropylethylamine or N-methylmorpholine or from the alkali metal carbonates or bicarbonates such as potassium carbonate, sodium carbonate or sodium bicarbonate. In the absence of a base, the reaction is carried out using an excess of the compound of formula (III) and in the presence of an alkali metal iodide such as potassium iodide or sodium iodide. The reaction is carried out at a temperature between room temperature and 100° C.

According to another variant of the method,

a compound of formula:

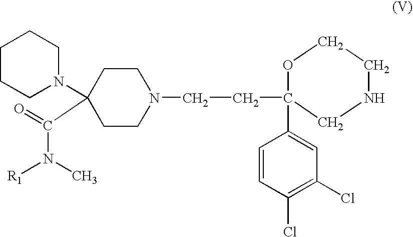

in which R₁ is as defined for a compound of formula (I), is reacted with a functional derivative of an acid of formula:

HOOC—B-Z (VI)

in which B and Z are as defined for a compound of formula (I).

Optionally, the compound of formula (I) is converted to one of its salts with inorganic or organic acids.

As a functional derivative of the acid (VI), the acid itself, or alternatively one of the functional derivatives which react with amines, for example an anhydride, a mixed anhydride, the acid chloride, or an activated ester, such as the para-nitrophenyl ester, is used.

When the acid of formula (VI) itself is used, the procedure is carried out in the presence of a coupling agent used in peptide chemistry such as 1,3-dicyclohexylcarbodiimine or benzotriazol-1-yloxy-tris(dimethylamino)phosphonium hexafluorophosphate in the presence of a base such as triethylamine or N,N-diisopropylethylamine, in an inert solvent such as dichloromethane or N,N-dimethylformamide at a temperature between 0° C. and room temperature.

When an acid chloride is used, the reaction is carried out in an inert solvent such as dichloromethane or benzene, in the presence of a base such as triethylamine or N-methylmorpholine and at a temperature between −60° C. and room temperature.

The compounds of formula (I) thus obtained may be subsequently separated from the reaction medium and purified according to conventional methods, for example by crystallization or chromatography.

The compounds of formula (I) thus obtained are isolated in the form of a free base or a salt, according to conventional techniques.

When the compounds of formula (I) are obtained in the form of a free base, the salification is carried out by treating with the chosen acid in an organic solvent. By treating the free base, dissolved for example in an ether such as diethyl ether or in an alcohol such as 2-propanol or in acetone or in dichloromethane, or in ethyl acetate or in acetonitrile with a solution of the chosen acid in one of the abovementioned solvents, the corresponding salt is obtained which is isolated according to conventional techniques.

Thus, the hydrochloride, hydrobromide, sulphate, trifluoroacetate, hydrogen sulphate, dihydrogen sulphate, methanesulphonate, oxalate, maleate, succinate, fumarate, naphthalene-2-sulphonate, benzenesulphonate, para-toluenesulphonate, gluconate, citrate or acetate is, for example, prepared.

At the end of the reaction, the compounds of formula (I) may be isolated in the form of one of their salts, for example, the hydrochloride or oxalate; in this case, if it is necessary, the free base may be prepared by neutralizing the said salt with an inorganic or organic base, such as sodium hydroxide or triethylamine or with an alkali metal carbonate or bicarbonate, such as sodium or potassium carbonate or bicarbonate.

The compounds of formula (II) are prepared according to known methods such as those described in WO 96/23787.

For example, a compound of formula (II) is prepared according to SCHEME 1 below in which E represents a hydrogen atom or an O-protecting group.

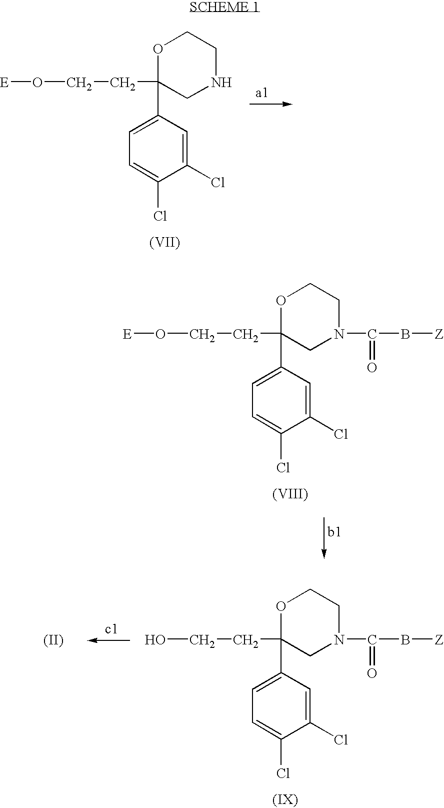

When E represents a protecting group, the latter is chosen from conventional O-protecting groups well known to a person skilled in the art, such as for example tetrahydropyran-2-yl, benzoyl or a (C₁-C₄) alkylcarbonyl.

In Step a1 of SCHEME 1, a compound of formula (VII) is reacted with a functional derivative of an acid of formula (VI), according to the methods previously described, in order to obtain a compound of formula (VIII).

The compound of formula (VIII) thus obtained is optionally deprotected in Step b1 according to methods known to a person skilled in the art. For example, when E represents a tetrahydropyran-2-yl group, the deprotection is carried out by acid hydrolysis using hydrochloric acid in a solvent such as ether, methanol or a mixture of these solvents, or using pyridinium p-toluenesulphonate in a solvent such as methanol or alternatively, using a resin Amberlyst® in a solvent such as methanol. The reaction is carried out at a temperature between room temperature and the reflux temperature of the solvent. When E represents a benzoyl group or a (C₁-C₄)alkylcarbonyl group, the deprotection is carried out by hydrolysis in alkaline medium using for example an alkali metal hydroxide such as sodium hydroxide, potassium hydroxide or lithium hydroxide, in an inert solvent such as water, methanol, ethanol, dioxane or a mixture of these solvents, at a temperature of between 0° C. and the reflux temperature of the solvent.

In Step c1, the alcohol of formula (IX) is oxidized in order to obtain the aldehyde of formula (II). The oxidation reaction is carried out using, for example, oxalyl chloride, dimethyl sulphoxide and triethylamine in a solvent such as dichloromethane and at a temperature of between −78° C. and room temperature.

The compounds of formula (III) are known and are prepared according to known methods. For example, a compound of formula (III) is prepared according to SCHEME 2 below.

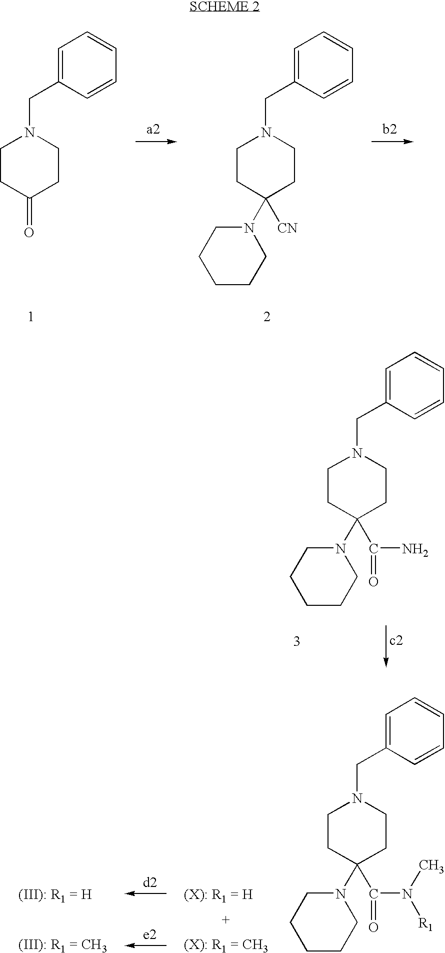

Steps a2 and b2 of SCHEME 2 are carried out according to the procedures described in Steps A and B of Preparation 2.16 in WO 96/23787.

In Step c2, compound 3 is reacted with a methyl halide, preferably methyl iodide, in the presence of a strong base such as sodium hydride, in a solvent such as tetrahydrofuran and at a temperature between room temperature and the reflux temperature of the solvent and a mixture of the compound of formula (X) in which R₁=H and the compound of formula (X) in which R₁=CH₃ is obtained which is separated according to conventional methods such as chromatography.

The compounds (X) are deprotected in Steps d2 or e2 according to known methods in order to give the expected compounds of formula (III).

The compounds of formula (IV) are prepared according to known methods such as those described in WO 96/23787. For example, a compound of formula (IX) is reacted with a compound of formula:

Y—SO₂—Cl (XI)

in which Y represents a methyl, phenyl, tolyl or trifluoromethyl group. The reaction is carried out in the presence of a base such as triethylamine, pyridine, N,N-diisopropylamine or N-methylmorpholine, in a solvent such as dichloromethane or toluene, and at a temperature of between −20° C. and the reflux temperature of the solvent.

The compounds of formula (V) are prepared according to SCHEME 3 below in which E represents hydrogen or an O-protecting group and Pr represents an N-protecting group.

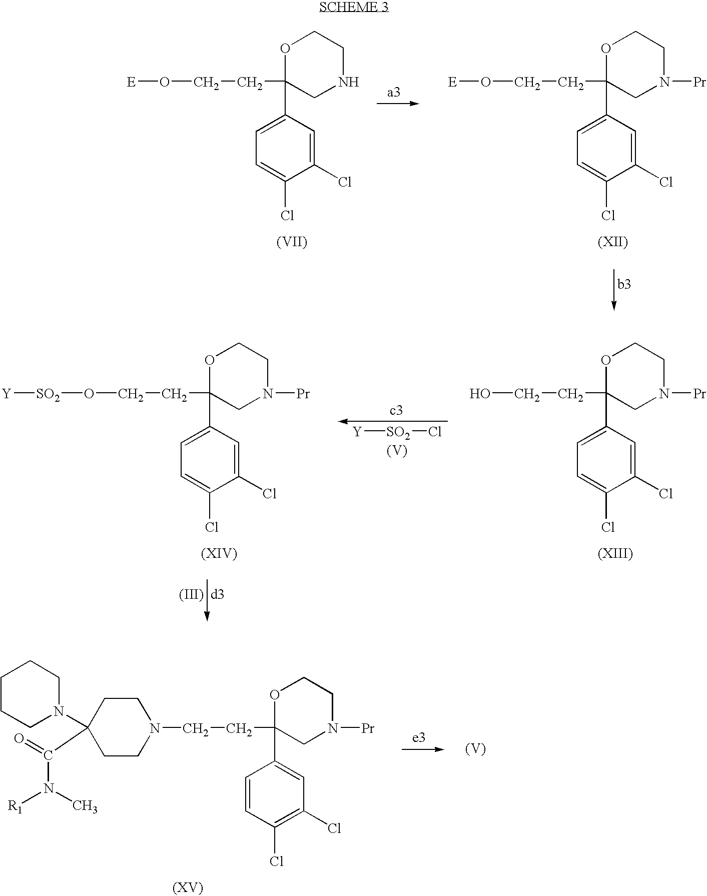

When Pr represents an N-protecting group, the latter is chosen from conventional N-protecting groups well known to a person skilled in the art such as, for example, the tert-butoxycarbonyl, benzyloxycarbonyl or trityl group.

The compounds of formula (VI) are marketed or prepared according to known methods. Thus, for example, 2-(2,3-dichlorophenyl)acetic acid is prepared according to SCHEME 4 below following the procedures described in Preparation 1.1.

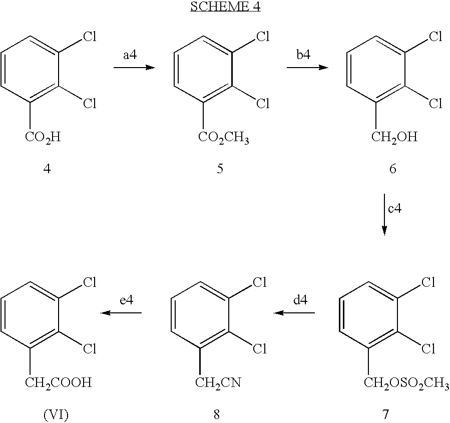

The compounds of formula (VII) are known and prepared according to known methods such as those described in WO 96/23787, in WO 01/04105, in WO 00/58292 or in Tetrahedron: Asymmetry, 1988, 9, 3251-3262.

During any one of the steps for preparing the compounds of formula (I) or the intermediate compounds of formula (II), (III), (IV), (V) or (VI), it may be necessary and/or desirable to protect the reactive or sensitive functional groups, such as the amine, hydroxyl or carboxyl groups, present on any one of the molecules involved. This protection may be carried out using conventional protecting groups such as those described in Protective Groups in Organic Chemistry, J. F. W. McOmie, ed. Plenum Press, 1973, in Protective Groups in Organic Synthesis, T. W. Greene and P. G. M. Wutts, Ed. John Wiley and Sons, 1991 or in Protecting Groups, Kocienski P. J., 1994, Georg Thieme Verlag. The elimination of the protecting groups may be carried out in an appropriate subsequent step using methods known to a person skilled in the art and which do not affect the rest of the molecule involved.

The resolution of the racemic mixtures of the compounds of formula (I) makes it possible to isolate the enantiomers.

It is however preferable to carry out the resolution of the racemic mixtures from the compound of formula (VII, E=H) or alternatively from an intermediate compound useful for preparing a compound of formula (VII), according to the methods described in the publications cited above for the preparation of a compound of formula (VII).

The compounds of formula (I) above also comprise those in which one or more hydrogen or carbon atoms have been replaced by their radioactive isotope, for example tritium, or carbon-14. Such labelled compounds are useful in research, metabolic or pharmacokinetic work, or in biochemical trials as receptor ligands.

The compounds according to the invention have been the subject of biochemical tests.

The affinity of the compounds for tachykinin receptors was evaluated in vitro by several biochemical tests using radioligands:

1) The binding of [¹²⁵I]BH-SP (Substance P labelled with iodine-125 using the Bolton-Hunter reagent) to the NK₁ receptors of human lymphoblastic cells (D. G. Payan et al., J. Immunol., 1984, 133, 3260-3265).

2) The binding of [¹²⁵I]|His-NKA to the cloned human NK₂ receptors expressed by CHO cells (Y. Takeda et al., J. Neurochem., 1992, 59, 740-745).

3) The binding of [¹²⁵I]His[MePhe⁷]NKB to the cloned human NK₃ receptors expressed by CHO cells (Buell et al., FEBS Letters, 1992, 299, 90-95).

The tests were carried out according to X. Emonds-Alt et al. (Eur. J. Pharmacol., 1993, 250, 403-413; Life Sci., 1995, 56, PL 27-32).

The compounds according to the invention weakly inhibit the binding of substance P to the NK₁ receptors of the human lymphoblastic cells IM9. The inhibition constant Ki for the receptors of the human lymphoblastic cells is greater than or equal to 8×10⁻⁹M.

The compounds according to the invention strongly inhibit the binding of [¹²⁵I]His-NKA to the cloned human NK₂ receptors. The inhibition constant Ki is less than or equal to 5×10⁻¹⁰M. Thus, the compound of Example 1 possesses a Ki equal to 4×10⁻¹¹M.

The compounds according to the invention strongly inhibit the binding of [¹²⁵I]His[MePhe⁷]NKB to the cloned human NK₃ receptors: the inhibition constant Ki is less than or equal to 7×10⁻¹M. Thus, the compound of Example 1 possesses a Ki equal to 4×10⁻¹M.

The prior art compound a inhibits the binding of [¹²⁵I]His-NKA to the cloned NK₂ receptors with a Ki equal to 4×10⁻¹M. It inhibits the binding of [¹²⁵I]His[MePhe⁷]NKB to the cloned human NK₃ receptors with a Ki equal to 2×10⁻⁹M.

The compounds of the present invention were also evaluated in vivo on animal models.

In gerbils, a rotating behaviour is induced by intrastriatal administration of a specific agonist of the NK₂ receptor, [Nle¹⁰]NKA(4-10); it was observed that a unilateral application of [Nle¹⁰]NKA(4-10) into the gerbil striatum leads to strong contralateral rotations which are inhibited by the compounds according to the invention administered either by the intraperitoneal route, or by the oral route. This test was carried out according to M. Poncelet et al., Neurosci, Lett., 1993, 149, 40-42. In this test, the compounds according to the invention are active at doses ranging from 0.1 mg to 30 mg per kg. For example, the compound of Example 1 possesses an effective dose 50 (ED₅₀) of 2.9 mg per kg by the intraperitoneal route and an ED₅₀ of 6.5 mg per kg by the oral route.

In gerbils, a rotating behaviour is induced by intrastriatal administration of a specific agonist of the NK₃ receptor: senktide; it is observed that a unilateral application of senktide into the gerbil striatum leads to strong contralateral rotations which are inhibited by the compounds according to the invention administered either by the intraperitoneal route, or by the oral route. This test was carried out according to X. Emonds-Alt et al., Life Sci., 1995, 56, PL27-PL32. In this test, the compounds according to the invention are active at doses ranging from 0.1 mg to 30 mg per kg. For example, the compound of Example 1 possesses an ED₅₀ of 2.8 mg per kg by the intraperitoneal route and an ED₅₀ of 4.3 mg per kg by the oral route.

In rats, the application of an agonist of the NK₂ receptors in the septum causes an increase in the release of acetylcholine in the hippocampus (test carried out according to R. Steinberg et al., Eur. J. Neurosci., 1998, 10, 2337-2345). Likewise in guinea pigs, the local application of an agonist of the NK₃ receptors in the septum causes an increase in the release of acetylcholine in the hippocampus (test carried out according to N. Marco et al., Neuropeptides, 1998, 32, 481-488). The compounds according to the invention block this increase in the release of acetylcholine whether it is caused by an agonist of the NK₂ receptors or by an agonist of the NK₃ receptors. For example, the compound of Example 1 blocks this increase in the release of acetylcholine caused either by an agonist of the NK₂ receptors in rats, or by an agonist of the NK₃ receptors in guinea pigs, at doses of 0.1-0.3 mg/kg and 0.3-1 mg/kg by the intraperitoneal route, respectively.

In rats, constraint stress causes an increase in the tissue level of DOPAC (3,4-dihydroxyphenyl acetic acid) in the prefrontal cortex (test carried out according to B. A. Morrow et al., Eur. J. Pharmacol., 1993, 238, 255-262). This increase is blocked by a specific antagonist of the NK₂ receptors such as saredutant (X. Emonds-Alt et al., Life Sci., 1992, 50, PL101-PL106) and is consequently mediated by the activation of the NK₂ receptors by the endogenous neurokinin A. It is observed that the compound of Example 1 administered at 1 mg/kg by the intraperitoneal route completely blocks this increase.

In guinea pigs, a treatment with haloperidol, administered at a dose of 1 mg/kg by the intraperitoneal route, causes an increase in the number of dopaminergic neurons which are spontaneously active (population response) in the A10 region (VTA, ventral tegmental area) of the brain, measured in electrophysiology. This increase is mediated by the activation of the NK₃ receptors by endogenous neurokinin B (C. Gueudet et al., Synapse, 1999, 33, 71-79). It is observed that the compound of Example 1 administered at 0.1-1 mg/kg by the intraperitoneal route blocks this increase.

All these pharmacological results show that the compounds according to the invention, in particular the compound of Example 1, are mixed antagonists of the NK₂ receptors and of the NK₃ receptors by blocking the pharmacological effects caused by neurokinin A or neurokinin B, whether they are applied exogenously or whether their endogenous release is provoked. Furthermore, these results show that the compounds according to the invention cross the blood-brain barrier well.

The compounds of the present invention are in particular active ingredients of pharmaceutical compositions, whose toxicity is compatible with their use as a medicament.

The compounds of formula (I) above may be used at daily doses of 0.01 to 100 mg per kilo of bodyweight of the mammal to be treated, preferably at daily doses of 0.1 to 50 mg/kg. In human beings, the dose may preferably vary from 0.1 to 4 000 mg per day, more particularly from 0.5 to 1 000 mg depending on the age of the subject to be treated or the type of treatment: prophylactic or curative.

For their use as medicaments, the compounds of formula (I) are generally administered in the form of dosage units. The said dosage units are preferably formulated in pharmaceutical compositions in which the active ingredient is mixed with one or more pharmaceutical excipients.

Thus, according to another of its aspects, the present invention relates to pharmaceutical compositions containing, as active ingredient, a compound of formula (I) or one of its pharmaceutically acceptable salts, solvates and/or hydrates.

In the pharmaceutical compositions of the present invention for administration by the oral, sublingual, inhaled, subcutaneous, intramuscular, intravenous, transdermal, local or rectal route, the active ingredients may be administered in unit forms for administration, in a mixture with conventional pharmaceutical carriers, to animals and to human beings. The appropriate unit forms for administration comprise the forms by the oral route such as tablets, gelatin capsules, powders, granules and oral solutions or suspensions, the forms for sublingual and buccal administration, aerosols, the forms for topical administration, implants, the forms for subcutaneous, intramuscular, intravenous, intranasal or intraocular administration and the forms for rectal administration.

When a solid composition is prepared in the form of tablets or gelatin capsules, there are added to the active ingredient, micronized or otherwise, a mixture of pharmaceutical excipients which may be composed of diluents such as for example lactose, microcrystalline cellulose, starch, dicalcium phosphate, binders such as for example polyvinylpyrrolidone, hydroxypropyl methyl cellulose, disintegrating agents such as crosslinked polyvinylpyrrolidone, crosslinked carboxymethyl cellulose, glidants such as silica, talc, lubricants such as magnesium stearate, stearic acid, glycerol tribehenate, sodium stearyl fumarate.

Wetting agents or surfactants such as sodium lauryl sulphate, polysorbate 80, poloxamer 188 may be added to the formulation.

The tablets may be prepared by various techniques, direct compression, dry granulation, wet granulation, hot-melt.

The tablets may be uncoated or coated with sugar (for example with sucrose) or coated with various polymers or other appropriate materials.

The tablets may have a flash, delayed or prolonged release by preparing polymeric matrices or using specific polymers in the film-coating.

The gelatin capsules may be soft or hard, film-coated or otherwise so as to have a flash, prolonged or delayed activity (for example by an enteric form).

They may not only contain a solid formulation formulated as above for the tablets, but also liquids or semisolids.

A preparation in syrup or elixir form may contain the active ingredient together with a sweetener, preferably calorie-free, methylparaben and propylparaben as antiseptic, as well as a taste enhancer and an appropriate colouring agent.

The powders or granules dispersible in water may contain the active ingredient in a mixture with dispersing agents, wetting agents or suspending agents, such as polyvinylpyrrolidone, as well as with sweeteners or flavour correctors.

For rectal administration, suppositories are used which are prepared with binders which melt at rectal temperature, for example cocoa butter or polyethylene glycols.

For parenteral, intranasal or intraocular administration, aqueous suspensions, isotonic saline solutions or sterile and injectable solutions are used which contain pharmacologically compatible dispersing agents and/or solubilizing agents, for example propylene glycol.

Thus, to prepare an aqueous solution which can be injected by the intravenous route, it is possible to use a cosolvent such as, for example, an alcohol such as ethanol or a glycol such as polyethylene glycol or propylene glycol, and a hydrophilic surfactant such as polysorbate 80 or poloxamer 188. To prepare an oily solution which can be injected by the intramuscular route, it is possible to solubilize the active ingredient with a triglyceride or a glycerol ester.

For local administration, creams, ointments, gels, collyria and sprays may be used.

For transdermal administration, it is possible to use patches in multilaminated form or with a reservoir in which the active ingredient may be in alcoholic solution, or sprays.

For administration by inhalation, an aerosol is used which contains for example sorbitan trioleate or oleic acid as well as trichlorofluoromethane, dichlorofluoromethane, dichlorotetrafluoroethane, Freon substitutes or any other biologically compatible propellant gas; it is also possible to use a system containing the active ingredient alone or combined with an excipient, in powdered form.

The active ingredient may also be provided in the form of a complex with a cyclodextrin, for example α,β,γ-cyclodextrin, 2-hydroxypropyl-β-cyclodextrin.

The active ingredient may also be formulated in the form of microcapsules or microspheres, optionally with one or more carriers or additives.

Among the prolonged-release forms which are useful in the case of chronic treatments, implants may be used. These may be prepared in the form of an oily suspension or in the form of a suspension of microspheres in an isotonic medium.

In each dosage unit, the active ingredient of formula (I) is present in quantities appropriate for the daily doses envisaged. In general, each dosage unit is suitably adjusted according to the dosage and the type of administration envisaged, for example tablets, gelatin capsules and the like, sachets, ampoules, syrups and the like, drops such that such a dosage unit contains from 0.1 to 1 000 mg of active ingredient, preferably from 0.5 to 250 mg before being administered one to four times a day.

Although these dosages are examples of average situations, there may be particular cases where higher or lower dosages are appropriate; such dosages also belong to the invention. According to the usual practice, the dosage appropriate for each patient is determined by the doctor according to the mode of administration, the age, the weight and the response of the said patient.

According to one of its aspects, the present invention relates to the use of the compounds of formula (I), or of one of their pharmaceutically acceptable salts, solvates and/or hydrates for the preparation of medicaments intended for treating any pathology where either neurokinin A and/or NK₂ receptors, or neurokinin B and/or NK₃ receptors, or both neurokinin A and neurokinin B and/or NK₂ and NK₃ receptors are involved.

According to another of its aspects, the present invention relates to the use of the compounds of formula (I) or of one of their pharmaceutically acceptable salts, solvates and/or hydrates for the preparation of medicaments intended for treating pathologies of the respiratory, gastrointestinal, urinary, immune and cardiovascular system and of the central nervous system as well as pain, migraine, inflammation, nausea and vomiting, and skin diseases.

For example and in a non-limiting manner, the compounds of formula (I) are useful:

as analgesics, in particular in the treatment of traumatic pain such as post-operative pain; neuralgia of the brachial plexus; chronic pain such as arthritic pain caused by osteoarthritis, rheumatoid arthritis or psoriatic arthritis; neuropathic pain such as post-herpetic neuralgia, trigeminal neuralgia, segmental or intercostal neuralgia, fibromyalgia, causalgia, peripheral neuropathy, diabetic neuropathy, neuropathies induced by a chemotherapy, AIDS-related neuropathies, occipital neuralgia, geniculate neuralgia or glossopharyngeal neuralgia; the illusory pain of amputees; various forms of headache such as chronic or acute migraine, temporomandibular pain, maxillary sinus pain, facial neuralgism or odontalgia; pain experienced by cancer sufferers; pain of visceral origin; gastrointestinal pain; pain caused by compression of a nerve, pain caused by intensive sporting activity; dysmenorrhoea; menstrual pain; pain caused by meningitis or arachnoiditis; musculoskeletal pain; pain in the lower back caused by a spinal stenosis, a prolapsed disc or sciatica; pain experienced by angina sufferers; pain caused by ankylosing spondylitis; pain associated with gout; pain associated with burns, cicatrization or pruriginous dermatosis; thalamic pain;

as anti-inflammatory agents, in particular for treating inflammation in asthma, influenza, chronic bronchitis (in particular chronic obstructive bronchitis and COPD (chronic obstructive pulmonary disease)), coughs, allergies, bronchospasm and rheumatoid arthritis; inflammatory diseases of the gastrointestinal system, for example Crohn's disease, ulcerative colitis, pancreatitis, gastritis, intestinal inflammation, disorders caused by non-steroidal anti-inflammatory agents, inflammatory and secretory effects caused by bacterial infections, for example caused by Clostridium difficile; inflammatory skin diseases, for example herpes and eczema; inflammatory bladder diseases such as cystitis and incontinence; ophthalmic inflammations such as conjunctivitis and vitreoretinopathy; dental inflammations such as gingivitis and periodontitis;

in the treatment of allergic diseases, in particular of the skin, such as urticaria, contact dermatitis, atopic dermatitis and respiratory diseases such as rhinitis;

in the treatment of diseases of the central nervous system, in particular psychoses such as schizophrenia, mania and dementia; cognitive disorders such as Alzheimer's disease, anxiety, AIDS-related dementia; diabetic neuropathies; depression; Parkinson's disease; drug dependency; substance abuse; consciousness disorders, sleeping disorders, disorders of the circadian rhythm, mood disorders and epilepsy; Down's syndrome; Huntington's chorea; stress-related somatic disorders; neurodegenerative diseases such as Pick's disease or Creutzfeldt-Jacob disease; disorders associated with panic, phobia or stress;

in the treatment of modifications of the permeability of the blood-brain barrier during inflammatory and autoimmune processes of the central nervous system, for example during AIDS-related infections;

as a muscle relaxant and antispasmodic agent;

in the treatment of acute or delayed and anticipated nausea and vomiting, for example nausea and vomiting induced by drugs such as the agents used in chemotherapy in the case of cancer; by radiation therapy during irradiation of the thorax or the abdomen in the treatment of cancer or carcinoidosis; by ingestion of poison; by toxins caused by metabolic or infectious disorders such as gastritis, or produced during a bacterial or viral gastrointestinal infection; during pregnancy; during vestibular disorders such as travel sickness, vertigo or Meniere's disease; in post-operative diseases; the nausea and vomiting induced by dialysis or by prostaglandins; by gastrointestinal obstructions; in reduced gastrointestinal motility; in visceral pain caused by myocardial infarction or peritonitis; in migraine; in altitude sickness; by ingestion of opiate analgesics such as morphine; in gastro-oesophageal reflux; in acidic indigestion or overconsumption of food or drink, in gastric acidity, regurgitation, and heartburn, for example episodic or nocturnal heartburn or heartburn induced by a meal and dyspepsia;

in the treatment of diseases of the gastrointestinal system such as irritable bowel syndrome, gastric and duodenal ulcers, oesophageal ulcers, diarrhoea, hypersecretions, lymphomas, gastritis, gastro-oesophageal reflux, faecal incontinence and Hirschsprung's disease;

in the treatment of skin diseases such as psoriasis, pruritus and burns, in particular sunburn;

in the treatment of diseases of the cardiovascular system such as hypertension, the vascular aspects of migraine, oedema, thrombosis, angina pectoris, vascular spasms, circulatory diseases caused by vasodilation, Raynaud's disease, fibrosis, collagen diseases and atherosclerosis, preeclampsia;

in the treatment of small-cell and large-cell lung cancers; breast cancer; cerebral tumours; adenocarcinomas of the urogenital sphere; in adjuvant treatment to prevent metastases;

demyelination diseases such as multiple sclerosis or amyotrophic lateral sclerosis;

in the treatment of diseases of the immune system associated with suppression or stimulation of the functions of the immune cells, for example rheumatoid arthritis, psoriasis, Crohn's disease, diabetes, lupus and rejection reactions after transplantation;

in the treatment of miction disorders, in particular pollakiuria, stress incontinence, urge incontinence, post-partum incontinence;

in the treatment of histiocytic reticulosis, for instance in lymphatic tissues;

as an anorexigenic agent;

in the treatment of emphysema; Reiter's disease; haemorrhoids;

in the treatment of ocular disorders such as glaucoma, ocular hypertension, myosis and excessive lachrymal secretion;

in the treatment or prevention of a stroke, epilepsy, cranial trauma, spinal cord trauma, cerebral ischaemic lesions caused by vascular attack or occlusion;

in the treatment of disorders of heart rate and cardiac rhythm, in particular those occasioned by pain or stress;

in the treatment of sensitive skin and for preventing or combating irritation of the skin or mucous membranes, dandruff, erythema or pruritus;

in the treatment of neurological skin disorders such as lichens, prurigo, pruriginous toxidermia and severe pruritus of neurogenic origin;

in the treatment of ulcers and of all diseases caused by Helicobacter pylori or a urease-positive gram-negative bacterium;

in the treatment of diseases caused by angiogenesis or in which angiogenesis is a symptom;

in the treatment of ocular and/or palpebral algia and/or ocular or palpebral dysesthesia;

as an antiperspirant.

The present invention also includes a method for treating the said complaints at the doses indicated above.

The pharmaceutical compositions according to the present invention can also contain other active products that are useful for treating the diseases or disorders indicated above, for example bronchodilators, antitussive agents, antihistamines, antiinflammatory agents, antiemetic agents and chemotherapy agents.

The following Preparations and Examples illustrate the invention without however limiting it.

The following abbreviations are used in the Preparations and in the Examples:

DMF: dimethylformamide

DMSO: dimethyl sulphoxide

DCM: dichloromethane

THF: tetrahydrofuran

hydrochloric ether: saturated solution of hydrochloric acid in ether

BOP: benzotriazol-1-yloxytris(dimethylamino)-phosphonium hexafluorophosphate

m.p.: melting point

RT: room temperature

b.p.: boiling point

silica H: 60H silica gel sold by Merck (Darmstadt).

The proton nuclear magnetic resonance (¹H NMR) spectra are recorded at 200 MHz in DMSO-d₆, using the DMSO-d₆ peak as reference. The chemical shifts δ are indicated in parts per million (ppm). The signals observed are expressed as follows:

s: singlet; se: broad singlet; t: triplet; qd: quartet;

m: unresolved complex; mt: multiplet.

The NMR spectra confirm the structures of the compounds.

Preparations

1. Preparation of the Compounds of Formula (VI).

Preparation 1.1

### 2-(2,3-Dichlorophenyl)acetic acid

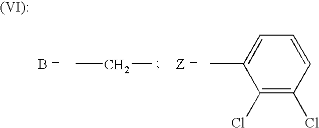

### A) Methyl ester of 2,3-dichlorobenzoic acid

6 ml of concentrated sulphuric acid are added to a solution of 25.08 g of 2,3-dichlorobenzoic acid in 125 ml of MeOH, and then the mixture is heated under reflux overnight. The reaction mixture is concentrated under vacuum, the residue is taken up in water, the medium is alkalinized by adding a 10% solution of NaHCO₃, and extracted with ether, the organic phase is washed twice with water, dried over Na₂SO₄ and the solvent is evaporated under vacuum. 25.68 g of the expected product are obtained.

### B) 2,3-Dichlorobenzyl alcohol

A suspension of 10.56 g of lithium aluminium hydride in 125 ml of THF is cooled to 0° C., a solution of 25.68 g of the compound obtained in the preceding step in 100 ml of THF is added dropwise, the temperature is allowed to return to RT and the mixture is kept stirred for 2 hours at RT. The reaction mixture is diluted by adding 250 ml of THF and hydrolysed by adding 11 ml of water, 11 ml of 4N NaOH and 33 ml of water. It is allowed to stand overnight at RT, the inorganic salts are filtered and the filtrate is concentrated under vacuum. 21.54 g of the expected product are obtained after drying under vacuum at 30° C.

### C) 2,3-Dichlorobenzyl methanesulphonate

A solution of 21.54 g of the compound obtained in the preceding step and 18.6 ml of triethylamine in 150 ml of DCM is cooled in an ice bath, a solution of 10.4 ml of methanesulphonyl chloride in 50 ml of DCM is added dropwise at a temperature of less than 10° C. and the mixture is kept stirred while allowing the temperature to return to RT. It is concentrated under vacuum, the residue is extracted with ether, and the medium is washed twice with a buffer solution pH=2, with a saturated solution of NaCl, dried over Na₂SO₄ and the solvent is evaporated under vacuum. 29.25 g of the expected product are obtained.

### D) 2,3-Dichlorophenylacetonitrile

10.1 g of potassium cyanide at 97% are added to a solution of 29.25 g of the compound obtained in the preceding step in 200 ml of EtOH and 50 ml of water and the mixture is heated under reflux for 2 hours. It is concentrated under vacuum, the residue is extracted with AcOEt, the organic phase is washed four times with water, with a saturated solution of NaCl, dried over Na₂SO₄ and the solvent is evaporated under vacuum. The residue is taken up in 200 ml of pentane and the medium is allowed to crystallize overnight, with stirring. The precipitate formed is drained and dried under vacuum. 17.17 g of the expected product are obtained.

### E) 2-(2,3-Dichlorophenyl)acetic acid

A solution of 24.23 g of KOH in 74 ml of water is added to a solution of 17.17 g of the compound obtained in the preceding step in 188 ml of EtOH, and then the mixture is heated overnight under reflux. It is concentrated under vacuum, the residue is taken up in 100 ml of water, the aqueous phase is washed three times with ether, the aqueous phase is acidified to pH=1 by adding a concentrated HCl solution, and allowed to crystallize, with stirring, by cooling in an ice bath. The precipitate formed is drained, washed with water and dried under vacuum at 40° C. 17.17 g of the expected product are obtained.

2. Preparation of the Compounds of Formula (II).

Preparation 2.1

### 2-[4-Benzoyl-2-(3,4-dichlorophenyl)morpholin-2-yl]acetaldehyde, sole isomer

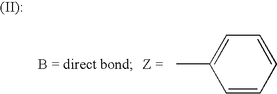

### A) 2-[2-(3,4-Dichlorophenyl)morpholin-2-yl]ethyl benzoate, laevorotatory isomer

This compound is prepared according to the procedure described in Preparation 1.1 in WO 00/58292.

### B) [2(3,4-dichlorophenyl)-2-(2-hydroxyethyl)-morpholin-4-yl](phenyl)methanone, sole isomer

A solution of 4 g of the compound obtained in the preceding step and 1.5 ml of triethylamine in 100 ml of DCM is cooled to 0° C., a solution of 1.41 g of benzoyl chloride in 10 ml of DCM is added dropwise and the mixture is kept stirred for 30 minutes. The reaction mixture is concentrated under vacuum, the residue is extracted with ether, the organic phase is washed with water, with a buffer solution pH=2, with water, with a saturated solution of NaCl, dried over Na₂SO₄ and the solvent is evaporated under vacuum. The oily residue thus obtained is taken up in 70 ml of 95% EtOH, 2.5 ml of a 30% NaOH solution are added and the mixture is kept stirred for 1 hour at RT. It is concentrated under vacuum, the residue is extracted with AcOEt, the organic phase is washed three times with water, with a saturated solution of NaCl, dried over Na₂SO₄ and the solvent is evaporated under vacuum. 4 g of the expected product are obtained.

### C) 2-[4-Benzoyl-2-(3,4-dichlorophenyl)morpholin-2-yl]acetaldehyde, sole isomer

A solution of 1.85 g of the compound obtained in the preceding step and 2.25 ml of DMSO in 25 ml of DCM is cooled to −60° C., under a nitrogen atmosphere, 1.38 ml of oxalyl chloride are added dropwise and the mixture is kept stirred for 2 hours at −60° C. 4.42 ml of triethylamine are then added and the mixture is kept stirred while allowing the temperature to return to RT. The reaction mixture is diluted by adding DCM, the organic phase is washed with water, with a 10% solution of Na₂CO₃, twice with water, with a saturated solution of NaCl, dried over Na₂SO₄ and the solvent is evaporated under vacuum. 1.7 g of the expected product are obtained.

Preparation 2.2

### 2-[4-(2,3-Dichlorobenzoyl)-2-(3,4-dichloro-phenyl)morpholin-2-yl]acetaldehyde, sole isomer

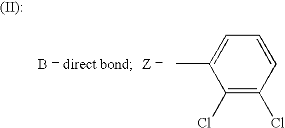

### A) (2,3-Dichlorophenyl)[2-(3,4-dichlorophenyl)-2-(2-hydroxyethyl)morpholin-4-yl]methanone, sole isomer

3.3 g of BOP are added to a solution of 2.5 g of the compound obtained in Step A) of Preparation 2.1, 1.2 g of 2,3-dichlorobenzoic acid and 0.75 g of triethylamine in 50 ml of DCM and the mixture is kept stirred for 30 minutes at RT. It is concentrated under vacuum, the residue is extracted with AcOEt, the organic phase is washed with water, with a buffer solution pH=2, with water, dried over Na₂SO₄ and the solvent is evaporated under vacuum. The residue is taken up in 30 ml of MeOH, 3 ml of a 30% NaOH solution are added, and the mixture is kept stirred for 30 minutes at RT. It is concentrated under vacuum, the residue is extracted with ether, the organic phase is washed with water, dried over Na₂SO₄ and the solvent is evaporated under vacuum. The residue is chromatographed on a silica gel H, eluting with the gradient of the DCM/MeOH mixture from (100/0.1; v/v) to (100/1; v/v). 1.55 g of the expected product are obtained.

### B) 2-[4-(2,3-Dichlorobenzoyl)-2-(3,4-dichlorophenyl)-morpholin-2-yl]acetaldehyde, sole isomer

A solution of 1.5 g of the compound obtained in the preceding step and 1.5 g of DMSO in 20 ml of DCM is cooled to −60° C., 1.25 g of oxalyl chloride are added dropwise and the mixture is kept stirred for 1 hour at −60° C. 2 g of triethylamine are then added and the mixture is kept stirred, allowing the temperature to return to RT. The reaction mixture is extracted with DCM, the organic phase is washed with a 1N HCl solution, with water, dried over Na₂SO₄ and the solvent is evaporated under vacuum. 1.4 g of the expected product are obtained.

Preparation 2.3

### 2-[2-(3,4-Dichlorophenyl)-4-[2-(2,6-dichlorophenyl)acetyl]morpholin-2-yl]acetaldehyde, sole isomer

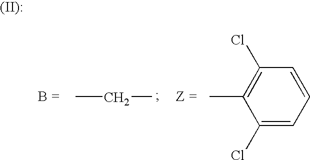

### A) 2-[2-(3,4-Dichlorophenyl)-4-[2-(2,6-dichlorophenyl)acetyl]morpholin-2-yl]ethyl benzoate, sole isomer

A solution of 4 g of the compound obtained in Step A of Preparation 2.1 in 43 ml of DCM is cooled to 0° C., 2.16 g of 2-(2,6-dichlorophenyl)acetic acid are added, followed by a solution of 3 ml of triethylamine in 50 ml of DCM and 4.7 g of BOP, and then the mixture is kept stirred while allowing the temperature to return to RT. It is concentrated under vacuum, the residue is extracted with AcOEt, the organic phase is washed with a 2N HCl solution, with water, with a 10% Na₂CO₃ solution, with water, with a saturated solution of NaCl, dried over Na₂SO₄ and the solvent is evaporated under vacuum. 6 g of the expected product are obtained.

### B) 2-(2,6-Dichlorophenyl)-1-[2-(3,4-dichlorophenyl)-2-(2-hydroxyethyl)morpholin-4-yl]-1-ethanone, sole isomer

A mixture of 6 g of the compound obtained in the preceding step in 100 ml of MeOH is heated under reflux, 3.5 ml of a 30% NaOH solution are added and the mixture is kept under reflux for 1 hour, with stirring. It is concentrated under vacuum, the residue is taken up in water, extracted with AcOEt, the organic phase is washed twice with water, with a saturated solution of NaCl, dried over Na₂SO₄ and the solvent is evaporated under vacuum. The residue is chromatographed on a silica gel H, eluting with DCM and then with the gradient of the DCM/MeOH mixture from (100/1; v/v) to (100/3; v/v). 2.42 g of the expected product are obtained.

### C) 2-[2-(3,4-Dichlorophenyl)-4-[2-(2,6-dichlorophenyl)acetyl]morpholin-2-yl]acetaldehyde, sole isomer

A mixture of 0.6 ml of oxalyl chloride in 11 ml of DCM is cooled to −60° C., a solution of 1.2 ml of DMSO in 5 ml of DCM is added, followed dropwise by a solution of 2.42 g of the compound obtained in the preceding step and 1.6 ml of DMSO in 11 ml of DCM and the mixture is kept stirred for 30 minutes at −50° C. 4.6 ml of triethylamine are then added and the mixture is kept stirred while allowing the temperature to return to RT. The reaction mixture is extracted with DCM, the organic phase is washed with a 2N HCl solution, with water, with a 10% Na₂CO₃ solution, with water, with a saturated solution of NaCl, dried over Na₂SO₄ and the solvent is evaporated under vacuum. 2.24 g of the expected product are obtained.

Preparation 2.4

### 2-[2-(3,4-Dichlorophenyl)-4-[2-(2,3-dichlorophenyl)acetyl]morpholin-2-yl]acetaldehyde, sole isomer

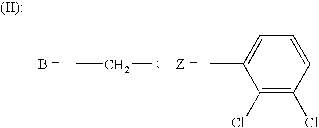

### A) 2-[2-(3,4-Dichlorophenyl)-4-[2-(2,3-dichlorophenyl)acetyl]morpholin-2-yl]ethyl benzoate, sole isomer

This compound is prepared according to the procedure described in Step A of Preparation 2.3 from 4.9 g of the compound obtained in Step A of Preparation 2.1 in 52 ml of DCM, 2.67 g of the compound obtained in Preparation 1.1, a solution of 3.62 ml of triethylamine in 36 ml of DCM and 5.76 g of BOP. 7.11 g of the expected product are obtained.

### B) 2-[2,3-Dichlorophenyl)-1-[2-(3,4-dichlorophenyl)-2-(2-hydroxyethyl)morpholin-4-yl]-1-ethanone, sole isomer

5 ml of a 30% NaOH solution are added to a solution of 7.11 g of the compound obtained in the preceding step in 100 ml of MeOH and the mixture is kept stirred for 1 hour at RT. It is concentrated under vacuum, the residue is extracted with AcOEt, the organic phase is washed twice with water, with a saturated NaCl solution, dried over Na₂SO₄ and the solvent is evaporated under vacuum. The residue is chromatographed on silica gel H, eluting with DCM and then with the DCM/MeOH mixture (100/1; v/v). 2.21 g of the expected product are obtained.

### C) 2-[2-(3,4-Dichlorophenyl)-4-(2-(2,3-dichlorophenyl)acetyl]morpholin-2-yl]acetaldehyde, sole isomer

This compound is prepared according to the procedure described in Step C of Preparation 2.3 from 0.5 ml of oxalyl chloride in 10 ml of DCM, a solution of 1.02 ml of DMSO in 5 ml of DCM, a solution of 2.21 g of the compound obtained in the preceding step and 1.43 ml of DMSO in 10 ml of DCM and 4.2 ml of triethylamine. 2.1 g of the expected product are obtained.

3. Preparation of the Compounds of Formula (III).

Preparation 3.1

### N,N-Dimethyl-4-(piperidin-1-yl)piperidine-4-carboxamide

R₁=—CH₃. (III).

### A) 1-Benzyl-4-cyano-4-(piperidin-1-yl)piperidine.

A solution of 5.3 g of sodium cyanide in 20 ml of water is added dropwise and at RT to a solution of 18.6 g of 1-benzylpiperidin-4-one and 12.16 g of piperidine hydrochloride in 25 ml of MeOH and 25 ml of water and the mixture is kept stirred for 48 hours at RT. The precipitate formed is drained, washed with water and dried under vacuum. 27 g of the expected product are obtained.

### B) 1-Benzyl-4-(piperidin-1-yl)piperidine-4-carboxamide

28.3 g of the compound obtained in the preceding step are added to 80 ml of 95% sulphuric acid and the mixture is heated at 100° C. for 10 minutes. After cooling to RT, the reaction mixture is poured over ice, brought to pH=7 by adding a 25% NH₄OH solution, extracted with DCM, the organic phase is washed with water, with a saturated solution of NaCl, dried over Na₂SO₄ and the solvent is evaporated under vacuum. The residue is taken up in acetone, kept stirred for 2 hours at RT and the precipitate formed is drained. 20.8 g of the expected product are obtained.

### C) N,N-Dimethyl-1-benzyl-4-(piperidin-1-yl)-piperidine-4-carboxamide and N-methyl-1-benzyl-4-(piperidin-1-yl)piperidine-4-carboxamide

A solution of 9.87 g of the compound obtained in the preceding step in 120 ml of THF is added dropwise and at RT to a suspension of 3.6 g of sodium hydride at 60% in oil in 120 ml of THF and the mixture is heated at 60° C. for 2 hours. After cooling to RT, a solution of 8.52 g of methyl iodide in 60 ml of DMF is added dropwise and the mixture is kept stirred for 4 hours at RT. The reaction mixture is poured over ice, extracted with ether, the organic phase is washed with water, dried over Na₂SO₄ and the solvent is evaporated under vacuum. The residue is chromatographed on silica gel H, eluting with the DCM/MeOH/NH₄OH mixture (100/1/0.1; v/v/v) and the following are separated:

the least polar compound: 6 g of N,N-dimethyl-1-benzyl-4-(piperidin-1-yl)piperidine-4-carboxamide are obtained;

the most polar compound: 2.6 g of N-methyl-1-benzyl-4-(piperidin-1-yl)piperidine-4-carboxamide are obtained.

### D) N,N-Dimethyl-4-(piperidin-1-yl)piperidine-4-carboxamide

A mixture of 5.9 g of the least polar compound obtained in the preceding step, 3.4 g of ammonium formate and 1.5g of 10% palladium on carbon in 60 ml of MeOH is kept stirred for 3 hours at RT. The catalyst is filtered on Celite® and the filtrate is concentrated under vacuum. 1.9 g of the expected product are obtained after drying under vacuum at 60° C.

Preparation 3.2

### N-Methyl-4-(piperidin-1-yl)piperidine-4-carboxamide formate

HCOOH: R₁=H. (III)

A mixture of 4 g of the most polar compound obtained in Step C of Preparation 3.1, 2.43 g of ammonium formate and 1 g of 10% palladium on carbon in 50 ml of MeOH is kept stirred for 30 minutes at RT. The catalyst is filtered on Celite® and the filtrate is concentrated under vacuum. 2.6 g of the expected product are obtained after drying-under vacuum.

### EXAMPLE 1

### N,N-Dimethyl-1-[2-[4-benzoyl-2-(3,4-dichlorophenyl)morpholin-2-yl]ethyl]-4-(piperidin-1-yl)-piperidine-4-carboxamide dihydrochloride, dextrorotatory isomer

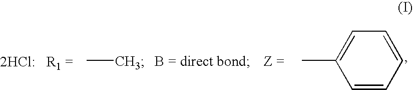

0.6 g of the compound obtained in Preparation 3.1 is added to a solution of 0.8 g of the compound obtained in Preparation 2.1 in 15 ml of DCM, followed by 0.9 g of sodium triacetoxyborohydride and 8 drops of acetic acid and the mixture is kept stirred overnight at RT. The reaction mixture is alkalinized by adding a 10% Na₂CO₃ solution, extracted with DCM, the organic phase is washed three times with water, with a saturated solution of NaCl, dried over Na₂SO₄ and the solvent is evaporated under vacuum. The residue is chromatographed on silica gel H, eluting with the gradient of the DCM/MeOH mixture from (100/0.5; v/v) to (100/2; v/v). The product obtained is taken up in hydrochloric ether and the solvent is evaporated under vacuum. 0.45 g of the expected product is obtained after crystallization from the pentane/iso ether mixture.

α_{D}²⁰=+14.4° (c=0.25; MeOH).

¹H NMR: DMSO-d₆+TFA, 350° K. δ(ppm): 1.3 to 1.8: m: 6H; 2.0 to 3.3: m: 20H; 3.3 to 4.2: m: 8H; 7.2 to 7.7: m: 8H.

### EXAMPLE 2

### N-Methyl-1-[2-[4-benzoyl-2-(3,4-dichlorophenyl)morpholin-2-yl]ethyl]-4-(piperidin-1-yl)piperidine-4-carboxamide dihydrochloride, dextrorotatory isomer

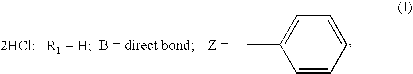

The compound is prepared according to the procedure described in Example 1 from 0.58 g of the compound obtained in Preparation 2.1, 15 ml of DCM, 0.345 g of the compound obtained in Preparation 3.2, 0.65 g of sodium triacetoxyborohydride and 8 drops of acetic acid. 0.6 g of the expected product is obtained after crystallization from the pentane/iso ether mixture.

α_{D}²⁰=+13.6° (c=0.25; MeOH).

### EXAMPLE 3

### N,N-Dimethyl-1-[2-[4-(2,3-dichlorobenzoyl)-2-(3,4-dichlorophenyl)morpholin-2-yl]ethyl]-4-(piperidin-1-yl)piperidine-4-carboxamide dihydrochloride, laevorotatory isomer

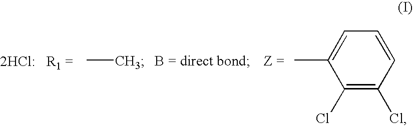

This compound is prepared according to the procedure described in Example 1 from 0.75 g of the compound obtained in Preparation 2.2, 20 ml of DCM, 0.43 g of the compound obtained in Preparation 3.1, 0.7 g of sodium triacetoxyborohydride and 8 drops of acetic acid. 0.8 g of the expected product is obtained after crystallization from the DCM/ether mixture.

α_{D}²⁰=−5.4° (c=0.5; MeOH).

### EXAMPLE 4

### N,N-Dimethyl-1-[2-[4-(2,6-dichlorophenyl)-acetyl]-2-(3,4-dichlorophenyl)morpholin-2-yl]ethyl]-4-(piperidin-1-yl)piperidine-4-carboxamide dihydrochloride, dextrorotatory isomer

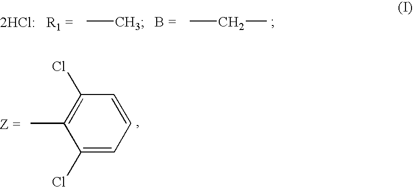

This compound is prepared according to the procedure described in Example 1 from 0.45 g of the compound obtained in Preparation 2.3, 50 ml of DCM, 0.28 g of the compound obtained in Preparation 3.1, 0.424 g of sodium triacetoxyborohydride and 3 drops of acetic acid. 0.419 g of the expected product is obtained after crystallization from ether.

α_{D}²⁰=+7.6° (c=0.25; MeOH).

### EXAMPLE 5

### N,N-Dimethyl-1-1-[2-[4-[2-(2,3-dichlorophenyl)-acetyl]-2-(3,4-dichlorophenyl)morpholin-2-yl]ethyl]-4-(piperidin-1-yl)piperidine-4-carboxamide dihydrochloride, dextrorotatory isomer, dihydrate

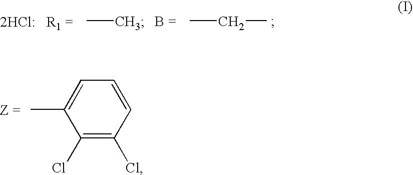

This compound is prepared according to the procedure described in Example 1 from 0.5 g of the compound obtained in Preparation 2.4, 7 ml of DCM, 0.312 g of the compound obtained in Preparation 3.1, 0.47 g of sodium triacetoxyborohydride and 3 drops of acetic acid. 0.446 g of the expected product is obtained after crystallization from ether.

α_{D}²⁰=+8.8° (c=0.25; MeOH).

### EXAMPLE 6

### N-Methyl-1-[2-[4-[2-(2,3-dichlorophenyl)-acetyl]-2-(3,4-dichlorophenyl)morpholin-2-yl]ethyl]-4-(piperidin-1-yl)piperidine-4-carboxamide dihydrochloride, dextrorotatory isomer, dihydrate

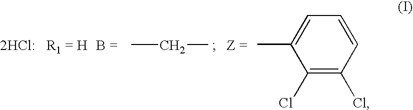

This compound is prepared according to the procedure described in Example 1 from 0.6 g of the compound obtained in Preparation 2.4, 60 ml of DCM, 0.3 g of the compound obtained in Preparation 3.2, 0.56 g of sodium triacetoxyborohydride and 3 drops of acetic acid. 0.556 g of the expected product is obtained after crystallization from ether.

α_{D}²⁰=+8° (c=0.25; MeOH).

1. Compound of formula: 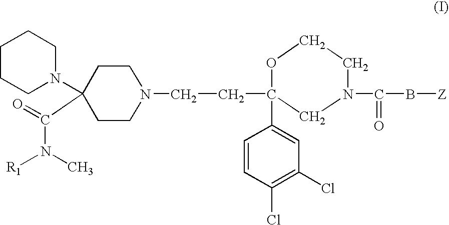
in which:
R₁ represents a hydrogen atom or a methyl radical;
B represents a direct bond or a —CH₂— group;
Z represents a phenyl, a 2,3-dichlorophenyl or a 2,6-dichlorophenyl;
and its salts with inorganic or organic acids, its solvates and/or its hydrates.

2. Compound of formula (I) according to claim 1, in the form of optically pure isomers.

3. Compound according to claim 1 or 2, chosen from:
N,N-dimethyl-1-[2-[4-benzoyl-2-(3,4-dichlorophenyl)-morpholin-2-yl]ethyl]-4-(piperidin-1-yl)piperidine-4-carboxamide, dextrorotatory isomer;
N-methyl-1-[2-[4-benzoyl-2-(3,4-dichlorophenyl)-morpholin-2-yl]ethyl]-4-(piperidin-1-yl)piperidine-4-carboxamide, dextrorotatory isomer;
N,N-dimethyl-1-[2-[4-2,3-dichlorobenzoyl)-2-(3,4-dichlorophenyl)morpholin-2-yl]ethyl]-4-(piperidin-1-yl)piperidine-4-carboxamide, laevorotatory isomer;
N,N-dimethyl-1-[2-[4-(2,6-dichlorophenyl)acetyl]-2-(3,4-dichlorophenyl)morpholin-2-yl]ethyl]-4-(piperidin-1-yl)piperidine-4-carboxamide, dextrorotatory isomer;
N,N-dimethyl-1-[2-[4-[2-(2,3-dichlorophenyl)acetyl]-2-(3,4-dichlorophenyl)morpholin-2-yl]ethyl]-4-(piperidin-1-yl)piperidine-4-carboxamide, dextrorotatory isomer;
N-methyl-1-[2-[4-[2-(2,3-dichlorophenyl)acetyl]-2-(3,4-dichlorophenyl)morpholin-2-yl]ethyl]-4-(piperidin-1-yl)piperidine-4-carboxamide, dextrorotatory isomer;
and its salts with inorganic or organic acids, its solvates and/or its hydrates.

4. Compound according to any one of claims 1 to 3, which is:
N,N-dimethyl-1-[2-[4-benzoyl-2-(3,4-dichlorophenyl)-morpholin-2-yl]ethyl]-4-(piperidin-1-yl)piperidine-4-carboxamide, dextrorotatory isomer;
and its salts with inorganic or organic acids, its solvates and/or its hydrates.

5. Method for preparing the compounds of formula (I) according to claim 1, their salts, their solvates and/or their hydrates, characterized in that: a compound of formula: 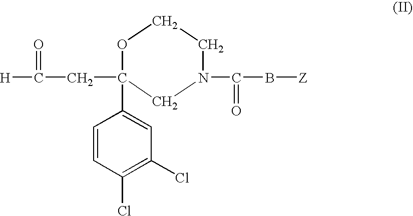
in which B and Z are as defined for a compound of formula (I) according to claim 1, is reacted with a compound of formula: 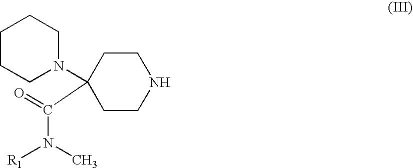
in which R₁ is as defined for a compound of formula (I) according to claim 1, in the presence of an acid, in a solvent, and then the intermediate iminium salt formed is reduced by means of a reducing agent.

6. Method for preparing the compounds of formula (I) according to claim 1, their salts, their solvates and/or their hydrates, characterized in that: a compound of formula: 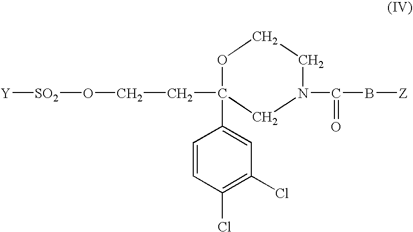
in which B and Z are as defined for a compound of formula (I) in claim 1 and Y represents a methyl, phenyl, tolyl or trifluoromethyl group, is reacted with a compound of formula: 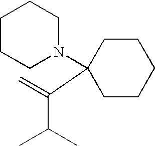
in which R₁ is as defined for a compound of formula (I) in claim 1.

7. Method for preparing the compounds of formula (I) according to claim 1, their salts, their solvates and/or their hydrates, characterized in that: a compound of formula: 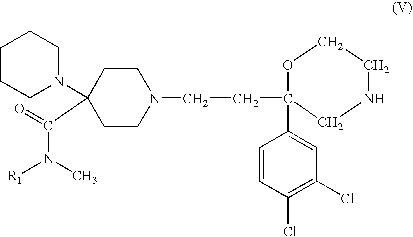
in which R₁ is as defined for a compound of formula (I) according to claim 1, is reacted with a functional derivative of an acid of formula:
HOOC—B-Z (VI)
in which B and Z are as defined for a compound of formula (I) according to claim 1.

8. Pharmaceutical composition comprising, as active ingredient, a compound according to any one of claims 1 to 4 or one of its pharmaceutically acceptable salts, solvates and/or hydrates.

9. Pharmaceutical composition according to claim 8, containing from 0.1 to 1 000 mg of active ingredient in dosage unit form in which the active ingredient is mixed with at least one pharmaceutical excipient.

10. Use of a compound according to any one of claims 1 to 4 or one of its pharmaceutically acceptable salts, solvates and/or hydrates for the preparation of medicaments intended for treating any pathology where either neurokinin A and/or NK₂ receptors, or neurokinin B and/or NK₃ receptors, or both neurokinin A and neurokinin B and/or NK₂ and NK₃ receptors are involved.

11. Use according to claim 10, for the preparation of medicaments intended for treating pathologies of the respiratory, gastrointestinal, urinary, immune and cardiovascular system and of the central nervous system as well as pain, migraine, inflammation, nausea and vomiting, and skin diseases.

12. Use according to claim 11, for preparing medicaments intended for treating chronic obstructive bronchitis, asthma, urinary incontinence, irritable bowel syndrome, Crohn's disease, ulcerative colitis, depression, anxiety, epilepsy, schizophrenia.

13. Medicament characterized in that it comprises a compound according to any one of claims 1 to 4 or one of its pharmaceutically acceptable salts, solvates and/or hydrates.
